(19) United States
(12) Patent Application Publication
(10) Pub. No.: US 2025/0262035 A1
(43) Pub. Date: Aug. 21, 2025

(54) Process for Producing a Blank for Dental Restorations by Way of a One-Step Sedimentation Process

(71) Applicant: Vita Zahnfabrik H. Rauter GmbH & Co. KG, Bad Säckingen (DE)

(72) Inventors: Andreas Oberländer, Bad Säckingen (DE); Berit Gödiker, Bad Säckingen (DE); Enno Bojemüller, Bad Säckingen (DE); Christian Strasser, Bad Säckingen (DE); Eduard Hellmehl, Bad Säckingen (DE)

(21) Appl. No.: 18/559,644

(22) PCT Filed: May 19, 2022
(86) PCT No.: PCT/EP2022/063577

§ 371 Date: May 23, 2024

(30) Foreign Application Priority Data
May 19, 2021 (EP) 21174713.4

(51) Int. Cl. A61C 13/00; A61K 6/17; A61K 6/60; A61K 6/78

(52) U.S. Cl. CPC A61C 13/0022; A61K 6/17; A61K 6/60; A61K 6/78; A61C 2201/002

(57) ABSTRACT

The present invention relates to a process for producing a color-graded blank for dental restorations by way of a sedimentation process, to a blank obtained by the process according to the invention and to the use of a suspension for producing a color-graded dental restoration. a) providing a suspension comprising i) a liquid dispersant, ii) a base material and iii) one or more coloring substances; b) sedimentation with the formation of a color gradient in the sediment; c) solidifying the sediment with the formation of a color-graded blank.

The present application relates to a process for producing a blank with a color gradient for dental restorations by way of a sedimentation process, a blank obtained by way of the process according to the invention, and the use of a suspension for producing a dental restoration with a color gradient.

The term “dental restoration” is supposed to mean measures for restoring defect teeth, or replacing lost teeth. This may be done, for example, in the form of fixed dental prostheses, such as crowns, partial crowns, bridges or implants, or in the form of removable denture, such as full or partial prostheses. These measures have in common that the restorations employed are supposed to come as close as possible to the appearance of natural teeth, in order to generate an aesthetically appealing view.

In order to achieve an aesthetically appealing restoration, which integrates into a patient's natural dental environment, the restoration may be colored accordingly, for example, by painting. Alternatively, blanks that already have a corresponding color gradient, which imitates that of the natural tooth, may be used for producing dental restorations. In order to achieve this color gradient, the blank is mostly composed of several layers, which differ with respect to their coloring.

WO 2020/025795 describes a blank for a dental restoration, which has a first and a second layer, which are composed independently of one another of glass, glass ceramic or ceramic, wherein the first layer and the second layer differ in the color and form a boundary surface, which extends at an angle within the blank.

DE 197 14 178 relates to a process for producing a multicolored shaped body for further processing into dental restorations, in which at least two differently colored starting materials are filled into one pressing die, which essentially defines the shape of the shaped body, and compressed into a shaped body.

EP 1 900 341 discloses a multicolored molded object with superimposed layers for the manufacture of dental restorations, which comprises (a) at least two consecutive and differently colored primary layers and (b) at least two differently colored intermediate layers between at least two consecutive and differently colored primary layers, where the primary layers have a thickness of more than 0.5 mm, and the intermediate layers have a thickness of 0.1 to 0.5 mm, and where between these intermediate layers, a change of the color takes place along a direction, which is opposite to the direction of the change in color between the primary layers.

According to EP 3 215 820, a computer-implemented process for producing a dental restoration includes the steps: capturing an image of a tooth at a color depth that is based on a multiplicity of color values; positioning the image by recognizing, in the tooth image, a contiguous first tooth color region with color values within a predefined first range of different color values, and assigning the first tooth color region to a common first false color value; and recognizing, in a tooth image, a contiguous second tooth color region with color values within a predefined second range of different color values, and assigning it to a common second false color value; determining a tooth color structure based on the first and second false color values of said first and second color range, respectively, within the tooth image; providing information on a multicolored block that has a predetermined color shading, formed by at least one block color zone and a second block color zone, wherein said information includes data about a block color structure with respect to dimensions and/or positions of said first and second block color zones; matching the tooth color structure to said block color structure; and based on said matching, determining a position within the block in which the tooth color structure and the block color structure are similar within predetermined limits; and machining the dental restoration from the block in said determined position.

EP 3 527 166 describes a milling blank made of plastic or a plastic-based composite with a continuous color gradient for producing an indirect dental restoration, and a method for producing such a blank by mixing two differently colored pastes with continuous variation of the mixing ratio of the two pastes during the filling process.

US 2019/381769 A1 discloses an oxide-ceramic multilayer block based on zirconium dioxide with different concentrations of lanthanum oxide, aluminum oxide, magnesium oxide, and yttrium oxide. The different metal oxides are obtained by infiltration with lanthanum-containing solutions for matching the sintering shrinks of the individual layers. The multilayer block is prepared by a pressing method.

US 2012/0308837 A1 discloses a multijet 3D printing method of ceramic suspensions based on waxes or polymers. These suspensions or printing pastes are applied layer by layer on top of one another. However, in printing, the layers are cured before the new layer is applied.

US 2019/0247168 A1 discloses a CAD/CAM block with a continuous color gradient. To prepare the continuous color gradient, two differently colored resins are poured into one mold, and the mixing ratio is continuously changed during the filling to establish the color gradient.

WO 2020/231329 A1 discloses a 3D printing method of objects with variable transmissions. The products are realized by applying printing resins with particles of different densities.

US 2018/0072628 A1 discloses a zirconium dioxide-based blank for dental applications with a color gradient that is obtained by different sedimentations speeds of colored particles having different particle sizes.

WO 2015/055950 A1 discloses mixtures of yttrium-stabilized zirconium dioxide, which may be used for the production of dental implants.

The processes and methods known in the prior art for preparing aesthetically appealing dental restorations have in common that the color generation is very complicated, and in part trained personnel with long years of experience is necessary to achieve satisfactory results. In the cases in which the restorations are produced from correspondingly colored blanks, there is a problem in that the layerwise structure, which is necessary for achieving the color gradient, often leads to the different layers being recognizable also in the finished restoration, and thus a graduate transition between the individual color layers cannot be achieved.

Therefore, there is still a need for processes that allow for the production of aesthetically appealing dental restorations. Therefore, it has been the object of the present invention to provide a process by which dental restorations become accessible whose optical appearance corresponds to that of a natural tooth, and can be adapted in accordance with the patient's needs.

Surprisingly, it has been found that this object is achieved by a process in which the blank is obtained by means of sedimentation methods.

The sedimentation behavior of solids contained in a suspension can be represented by the Stokes equation, as known by those skilled in the art:

$${v}_{p} = \frac{2}{9} * \frac{{r}^{2} g ( {ρ}_{p} - {ρ}_{f )}}{η} ,$$

where
vₚ: sedimentation rate
r: radius of the sinking object
g: gravitational acceleration
ρₚ: density of the particle
ρ_{f}: density of the fluid
n: dynamic viscosity of the fluid

Within the scope of the present invention, it has been surprisingly found that this general principle can also be assumed in the production of dental restorations by means of sedimentation-based shaping processes, in order to obtain blanks with a continuous color gradient.

Therefore, in a first aspect, the present application relates to a process for producing a blank with a color gradient for dental restorations, comprising the steps of:
a) providing a suspension comprising
i) liquid dispersant,
ii) a base material, and
iii) one or more coloring substances;
b) subjecting the suspension to sedimentation to form a sediment with a color gradient;
c) solidifying the sediment to form a blank having a color gradient.

Surprisingly, it has been found that the process according to the invention yields blanks that map the natural color gradient of a tooth without color transitions being recognizable, which would occur, for example, in blanks prepared by a layerwise build-up. Without being bound by theory, it is assumed that the continuous formation of the color gradient is explained, in particular, by the different sedimentation behaviors of the particles contained in the suspension.

The broad applicability of the process according to the invention to different materials has proven to be another advantage thereof. Thus, all the materials commonly used in the field of dental restorations may be employed as base materials. Preferably, the base material is a solid, especially a solid of biocompatible materials, especially biocompatible inorganic materials. Therefore, an embodiment is preferred in which said base material is selected from the group consisting of silicate glass ceramics and silicate glasses, aluminum oxides, spinels, and zirconium oxides.

In a particularly preferred embodiment, said base material is selected from the group consisting of silicate glass ceramics and silicate glasses, especially feldspars, foids, lithium silicates, and leucite glass ceramics. In a further preferred embodiment, said base material is colored.

In the process according to the invention, the color gradient in the blank is achieved by adding coloring substances, wherein the latter can be combined according to need. In a preferred embodiment, the coloring substances are color pigments, preferably oxides of elements selected from Zr, Hf, Zn, Ti, Fe, Al, Si, Cr, Sb, Mn, Cd, Sn, Ca, Na, K, Mg, Ni, Co, Sc, Y, Ce, Er, Yb, Pr, Eu, Dy, Tb, and mixtures thereof.

Especially preferably, the coloring substances are selected from inorganic pigments, especially coloring inorganic pigments. Biocompatible inorganic oxides are usually employed as coloring pigments. Preferably, inorganic pigments are employed that have a melting temperature of above 1300° C., further preferably those which essentially do not lose their coloring properties by exposure to heat of temperatures up to 1300° C. Possible pigments are familiar to those skilled in the art. For example, ZrO₂ or TiO₂ may be employed as white pigments, iron oxides, such as Fe₂O₃, as a red pigment, and doped ZrO₂ may be employed as a yellow pigment.

The color pigments may be in different forms in the suspension. Thus, an embodiment is preferred in which the color pigments are present in a colloidal form, powder form, or as frits.

The coloring of the blank can be achieved by different techniques. In a preferred embodiment, the base material and the coloring substances are present as a mixture in the suspension.

Within the scope of the present invention, it has been surprisingly found that modifying the surface of the coloring substances can influence the sedimentation behavior thereof, and thus the color gradient of the blank can be controlled. Thus, the coloring substances may be encapsulated, for example. Therefore, alternatively, an embodiment is preferred in which surface-modified pigments are employed as coloring substances. For example, the surface modification can be achieved by subjecting the coloring substances to thermal treatment in the presence of a suitable encapsulation material, in which the encapsulation material is molten and thus encloses the pigments that remained intact.

Preferably, the encapsulation material that can enclose the coloring substances is inorganic. Silicate glasses have proven to be suitable encapsulation materials. In a preferred embodiment of the present invention, the coloring substances are encapsulated, wherein, in particular, the encapsulating material is an inorganic solid having a melting point of below 1300° C. Particularly suitable encapsulating materials are low-melting glasses or glass-forming mixtures.

In a preferred embodiment, the encapsulated coloring substances are obtained by mixing inorganic pigments with the encapsulating material, followed by melting the encapsulating material. The pigments embedded in the melt are subsequently cooled, and the frit thus obtained is comminuted to the desired extent.

In another preferred embodiment, the encapsulating material is selected to be compatible with the base material. In one embodiment, the coloring substances may also be encapsulated with base material. This suggests itself, in particular, when the base material is selected to have a melting point below the melting point of the coloring substances, so that the coloring properties of the coloring substances are essentially unaffected.

In a further preferred embodiment, the base material and the coloring substances are in an integrated form, for example, as a frit. Such a material can be obtained, for example, by melting the base material together with the coloring substance or substances, and the frit thus obtained is brought to the desired particle size by milling.

The creation of the color gradient in the blank obtainable by the process according to the invention is determined, in particular, by the different sedimentation behaviors of the components of the suspension, which can in turn be influenced by selecting a suitable dispersant. Therefore, an embodiment is preferred in which said dispersant is selected from the group consisting of water, organic solvents, especially alcohols, liquid polymerizable monomers, and mixtures thereof.

In an embodiment of the present invention, the base material and the coloring substances are solids, especially inorganic solids. The base material and the coloring substances are different, especially in terms of their chemical composition and/or their optical properties.

The sedimentation behavior of solids contained in a suspension is influenced, inter alia, by the solids content of the suspension. Within the scope of the process according to the invention, it has proven particularly advantageous if the solids content of the suspension employed is not more than 70% by volume, based on the total volume of the suspension, because a continuous color gradient in the blank can be obtained in this way. Therefore, an embodiment is preferred in which the suspension has a solids content of 15 to 65% by volume, preferably from 35 to 60% by volume, respectively based on the total volume of the suspension.

The color gradient in the blank obtained according to the invention can further be determined by the density of the particles contained in the suspension. It has proven advantageous if the base material has a density that deviates from the density of the coloring substances, since the natural color gradient of a tooth can be imitated in this way. Therefore, an embodiment is preferred in which the base material has a density that deviates from the density of the coloring substances. Preferably, the density of the base material is lower than the density of the coloring substances. In a further preferred embodiment, the base material has a density of 2 to 6 g/cm³, preferably from 2 to 3 g/cm³. Said one or more coloring substances that are employed in the suspension preferably have a density of from 3 to 13 g/cm³, more preferably from 4 to 8 g/cm³. The density can be determined by helium pycnometry, for example.

In a preferred embodiment of the present invention, the difference in density between the base material and the coloring substances is at least 5%, preferably at least 10%, more preferably at least 15%, or at least 20% and especially at least 30%, respectively based on the density of the base material. In specific embodiments, the difference in density may be from 20 to 500%, preferably from 30 to 400%, more preferably from 40 to 250%, respectively based on the density of the base material.

Preferably, the ratio of the densities of base material to coloring materials is below 1, more preferably below 0.95, or below 0.85, especially within a range of from 0.4 to 0.8, especially within a range of from 0.5 to 0.75.

Another factor that can be relied on for influencing the sedimentation behavior is the particle size of the components contained in the suspension. Within the scope of the present invention, it has surprisingly been found that by selecting the base material and coloring substances with suitable particle sizes, it can be avoided that visible transitions between the differently colored regions of the blank form. Therefore, an embodiment is preferred in which the base material and/or the coloring substances have a median particle size D50 of from 0.03 to 100 μm, preferably from 1 to 20 μm, as determined by means of static and/or dynamic light scattering. The D50 value of the particle size designates the number of particles that are larger and smaller than the stated value, and is therefore also referred to as the median particle size.

The particle size distribution of any powdery material is usually stated by using the values D10, D50 and D90, in which D10 designates the 10% of the particles that have a particle size smaller than the stated value, D50 represents the median particle size, and D90 designates the 90% of the particles that have a particle size below the stated value. The % values are based on the total volume of the particles.

In a preferred embodiment, the process according to the invention is characterized in that the base material has a particle size distribution in which the D10 value is a fifth, preferably half, of the D50 value. Further, the D90 value of the particle size distribution is four times, preferably two times, the D50 value. The particle size distribution of the base material is preferably expressed as a D10 value of 0.2*D50, preferably 0.5*D50, and a D90 value of 4*D50, preferably 2*D50.

In a preferred embodiment, the base material has a particle size distribution in which the D10 value is within a range of from 0.2*D50 to 0.5*D50, more preferably from 0.25*D50 to 0.4*D50.

Also preferred is an embodiment in which the base material has a particle size distribution in which the D90 value is within a range of from 2*D50 to 4*D50, more preferably from 2.5*D50 to 3.5*D50.

The coloring substances employed in the process according to the invention are preferably characterized by the following particle size distribution: D10 of 0.1*D50, preferably 0.4*D50, and D90 of 5*D50, preferably 2*D50. In this manner, a natural color gradient of the blank could be achieved, without an excessive color intensity occurring in the lower region of the blank.

In a preferred embodiment, the coloring substances have a particle size distribution in which the D10 value is within a range of from 0.1*D50 to 0.4*D50, more preferably from 0.2*D50 to 0.35*D50.

Also preferred is an embodiment in which the coloring substances have a particle size distribution in which the D90 value is within a range of from 2*D50 to 5*D50, more preferably from 2.5*D50 to 4*D50.

The sought color gradient in the blank could be further optimized by adding further components to the suspension. Therefore, an embodiment of the process according to the invention is preferred in which the suspension further includes additives, preferably wetting agents and/or dispersants. The wetting agent is preferably selected from the group of non-ionic surfactants. The dispersant is preferably selected from the group consisting of water glass, polyelectrolytes, surfactants, carboxylic acid esters, amines, amine alcohols, polycarbonates, polycarboxylic acid derivatives, silanes, silicate polycarbonates, and mixtures thereof.

The blank obtained by the process according to the invention is provided as a dental restoration, in which it has proven advantageous to achieve the shaping of the blank by filling the suspension into corresponding casting molds. Therefore, especially relating to the viscosity of the suspension, it is to be taken care that, on the one hand, the desired color gradient is achieved and, on the other, that a bubble-free filling of the casting mold is effected. Therefore, an embodiment is preferred in which the suspension has a viscosity of 1 mPas to 10⁵ mPas, preferably 1 mPas to 10³ mPas, as determined at 25° C. by means of a rheometer (e.g., Physica MCR 101 Rheometer from Anton Paar).

The process according to the invention provides for the subjecting of the provided suspension to sedimentation. “Sedimentation” within the meaning of the present invention is the deposition or setting of the solids contained in the suspension. The deposition of the particles depends on their sedimentation rate, wherein the particles having the highest sedimentation rate will deposit first, i.e., are provided lowermost in the blank obtained according to the invention.

In a preferred embodiment, the sedimentation can be effected by means of a centrifugal force.

Step c) of the process according to the invention provides for the solidification of the sediment obtained in step b). Such solidification may be effected, for example, by drying, or in cases where polymerizable monomers are employed as dispersants, by polymerizing the monomers. In the solidification in step c) of the process according to the invention, preferably a firmness is reached which allows the blank to be handled, for example, in order to detach it from the casting mold, or to process it by means of CAD/CAM methods.

In a preferred embodiment of the process according to the invention, it further includes a step d), in which the sediment solidified in step c) is sintered. In the cases where polymerizable monomers are employed as dispersants, the desired firmness is achieved already by polymerizing the monomers. This can be done, for example, under high pressure conditions, so that the sintering step is omitted. In the cases where a sintering step is provided, the sintering can be performed up to a firmness that allows for the further processing of the blank by CAD/CAM methods, or up to the final density that is sought for its use as a dental restoration.

In a further preferred embodiment, the process according to the invention further includes a step of infiltrating the blank. In an infiltration, the blank is preferably infiltrated with a liquid curable composition, which is cured after the infiltration is completed. The infiltration step is provided mainly for the cases in which no liquid polymerizable monomers are used as dispersants. The liquid curable composition preferably includes organic monomers and/or prepolymers, preferably based on acrylates and/or methacrylates. In another preferred embodiment, said liquid curable composition is a glass or a glass ceramic. In one embodiment, the infiltration step can be performed after the drying of the sediment. In another embodiment, the infiltration step can be preceded by a sintering step, in which the sintering seeks a density of the blank that is, for example, 65 to 85% of the theoretical density. Suitable compounds and methods are known to the skilled person from WO 02/076907 and WO 2010/029515.

In another preferred embodiment, said liquid curable composition is a glass or a glass ceramic.

Within the scope of the present invention, it has been surprisingly found that blanks for dental restorations can be obtained by subjecting corresponding dispersions to sedimentation. Therefore, the present invention further relates to a blank produced by the process according to the invention, wherein said blank preferably has a color gradient. In a particularly preferred embodiment, said blank is a blank that has been infiltrated with a liquid curable composition. Further preferred is an embodiment in which said curable composition has been cured. In a particularly preferred embodiment, said blank is a blank made of a ceramic-polymer composite with interpenetrating networks, which is also referred to as a hybrid ceramic in the dental branch.

The present invention further relates to the use of a suspension for producing a blank or a dental restoration, wherein said suspension includes a liquid dispersant, a base material, and one or more coloring substances, wherein said blank or said dental restoration is characterized in that it has a color gradient that imitates the natural appearance of a tooth. The suspension employed is preferably one as described above.

The blank obtained can be processed to a dental restoration. The dental restoration, which is obtained either by further processing the blank or directly by the use of the suspension according to the invention, preferably includes inlays, onlays, table tops, veneers, crowns, partial crowns, implant-supported denture, or bridges.

### EXAMPLES

The present invention shall be further described by means of the following Examples, which should by no means be understood as limiting the idea of the invention. The values stated in % by volume respectively relate to the total volume of the suspension.

### 1. Controlling the Color Gradient by the Particle Size

Uncolored feldspar powder with a particle size D50 of 3 μm was mixed with red and yellow inorganic pigments each having a median particle size D50 of 6 μm, and a white pigment having a median particle size D50 of 1 μm. The mixture was dispersed in water, a non-ionic surfactant and water glass by using a ball mill, wherein the solids content of the suspension obtained was 53% by volume. The suspension was poured into a plastic casting mold, allowed to sediment, and dried. The dried sediment was sintered to a shaped body.

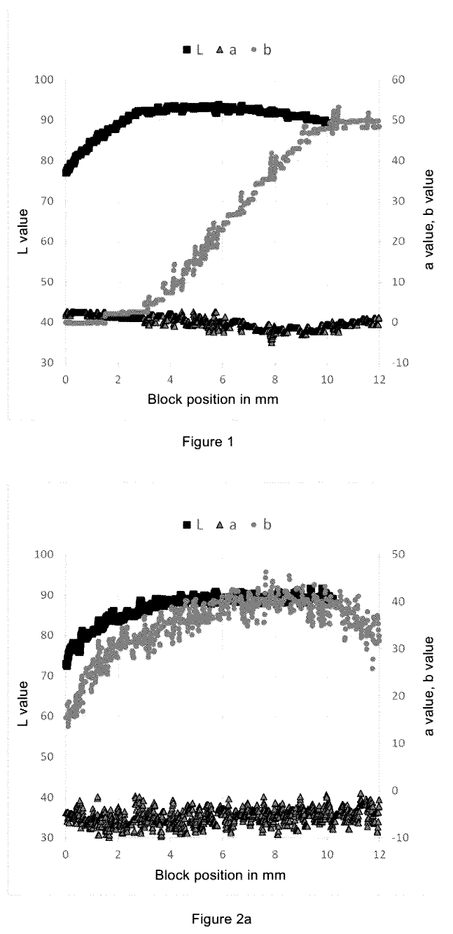

The color differences between the upper and lower regions of the blank obtained were clearly pronounced. Subsequent to a region of constant white coloring in the upper zone of the block, the yellow coloring increased continuously and constantly along the direction of sedimentation. FIG. 1 shows the LAB color values along the direction of sedimentation.

The color gradient of the blanks was tested using the Lab values (L*a*b). Thus, disks having a thickness of 3 mm were sawn from the specimens obtained, and photographs of these disks, positioned on a black background, were made using an optical microscope. Then, the color values along a profile were determined by using a corresponding image processing program.

### 2. Controlling the Color Gradient by the Solids Content

Uncolored feldspar powder with a particle size D50 of 3 μm was mixed with red and yellow pigments also having a particle size D50 of 3 μm, and a white pigment having a particle size D50 of 1 μm. The mixture was dispersed in water, a non-ionic surfactant and water glass by using a ball mill. In this manner, suspensions having a solids content of 52% by volume, 50% by volume and 46% by volume were prepared.

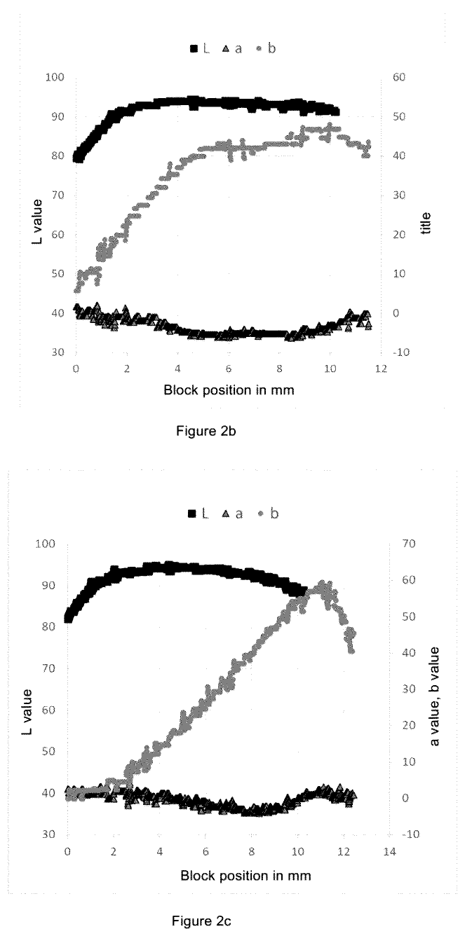

FIGS. 2a to c show the Lab values of the shaped bodies obtained from the suspensions by sedimentation, drying and sintering along the direction of sedimentation. As can be seen from the specimens, the color gradient of the shaped body increased as the solids content of the suspension decreased. Thus, the coloring of the blanks can be adjusted from monochrome to continuously polychrome in this way.

### 3. Surface-Modified Pigments

Red (Fe₂O₃), yellow (doped ZrO₂) and white (ZrO₂) pigments were respectively mixed with a silicate-based powder, and subjected to a thermal treatment in which said silicate-based powder and the pigments were melted into a frit, which was then comminuted to an average particle size by volume of 5 μm by milling.

The thus obtained powders were mixed together with an uncolored feldspar powder having a median volume-based particle size D50 of 3 μm, and the mixture was dispersed in water, a non-ionic surfactant and water glass by using a ball mill. From the thus produced suspensions, shaped bodies were obtained by sedimentation, drying and sintering, and the color gradient thereof was analyzed.

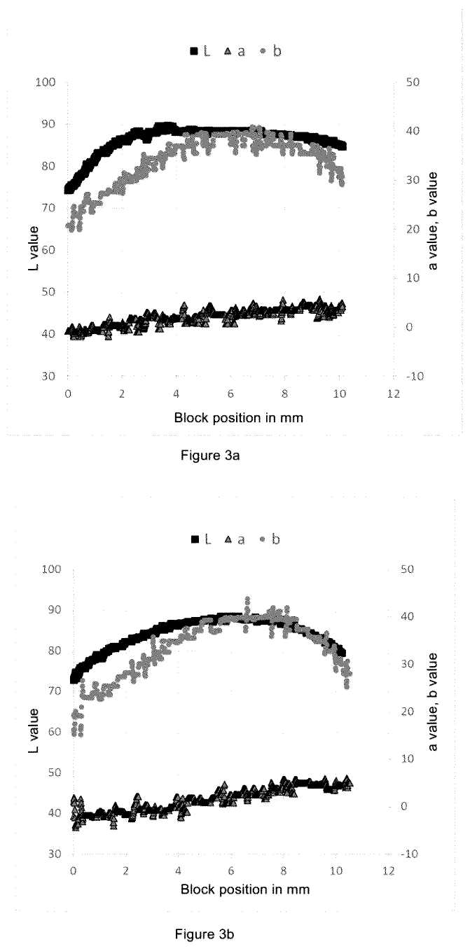

FIGS. 3a and b show the Lab color values of the blanks in the direction of sedimentation, wherein the solids content of the suspension was 50% by volume (FIG. 3a) and 48% by volume (FIG. 3b). As can be seen from the data made available, a significantly finer shading of the color gradient can be achieved by the encapsulation of the pigments. In addition to a continuous increase of the yellow proportion, the red coloring could also be selectively and continuously increased along the direction of sedimentation.

### 4. Precolored Base Material

Red, yellow and white pigments were mixed together with the uncolored base material, and thermally treated to obtain a frit. The frit was comminuted to the desired particle size by milling, and the powder was dispersed in water, a non-ionic surfactant and water glass by using a ball mill. The suspensions having a solids content of 52% by volume, 48% by volume and 46% by volume were sedimented, and the sediment obtained was dried and sintered. The color gradient of the thus obtained shaped bodies was measured along the direction of sedimentation.

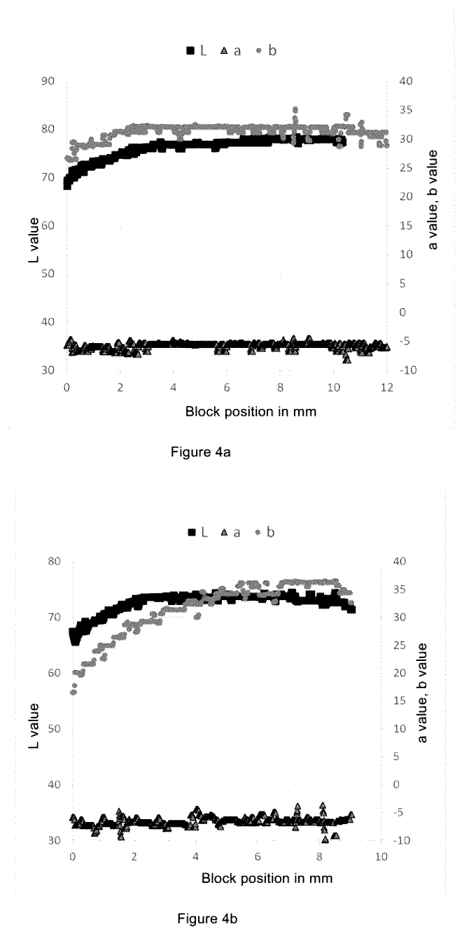

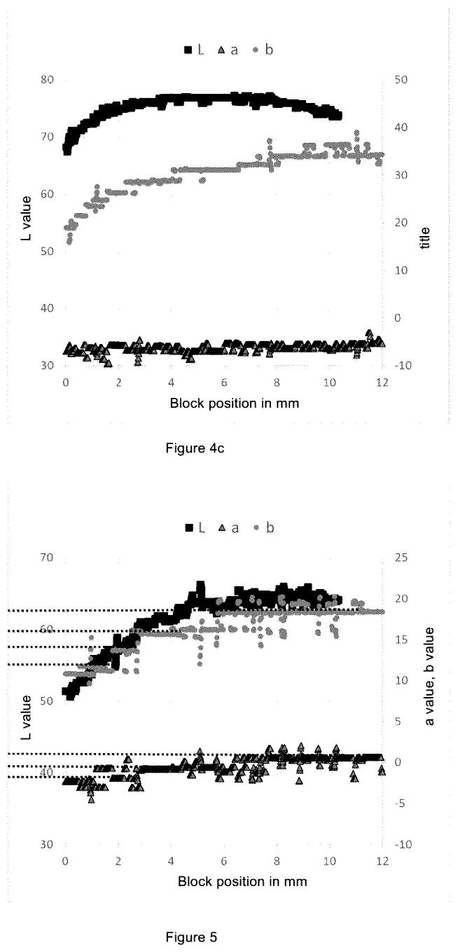

FIG. 4a shows the Lab values of the blank obtained from the suspension having a solids content of 52% by volume, FIG. 4b shows the one obtained from the suspension having a solids content of 48% by volume, and FIG. 4c shows the one obtained from the suspension having a solids content of 46% by volume.

As can be seen from the data made available, blanks having a very fine color gradient can be obtained in this way.

### 5. Comparative Example

A shaped body was produced by methods usual in the prior art, by preparing four mixtures of an uncolored base material and different concentrations of red pigment, yellow pigment, and white pigment. From these mixtures, four types of pressing granules were prepared by spray-drying, and compressed in a four-chamber feeder by using uniaxial multilayer pressing. The thus obtained shaped body was freed from binder and sintered, and the color gradient was measured. As can be seen from the Lab values shown in FIG. 5, the discontinuous changes of the color values are clearly visible despite the use of thin layers, which disturbs the natural appearance of the restoration material.

1. A process for producing a blank with a color gradient for dental restorations, comprising the steps of:
a) providing a suspension comprising
i) liquid dispersant,
ii) a base material, and
iii) one or more coloring substances;
b) subjecting the suspension to sedimentation to form a color gradient in the sediment;
c) solidifying the sediment to form a blank having a color gradient.

2. The process according to at least one of the preceding claims, characterized in that said base material is selected from the group consisting of silicate glass ceramics and silicate glasses, aluminum oxides, spinels, and zirconium oxides.

3. The process according to at least one of the preceding claims, characterized in that said coloring substances are selected from oxides of the elements Zr, Hf, Zn, Ti, Fe, Al, Si, Cr, Sb, Mn, Cd, Sn, Ca, Na, K, Mg, Ni, Co, Sc, Y, Ce, Er, Yb, Pr, Eu, Dy, Tb, and mixtures thereof.

4. The process according to at least one of the preceding claims, characterized in that the coloring substances are present in a colloidal form, powder form, or as frits.

5. The process according to at least one of the preceding claims, characterized in that said dispersant is selected from the group consisting of water, organic solvents, especially alcohols, liquid polymerizable monomers, and mixtures thereof.

6. The process according to at least one of the preceding claims, characterized in that the suspension has a solids content of 15 to 65% by volume, preferably from 35 to 60% by volume, respectively based on the total volume of the suspension.

7. The process according to at least one of the preceding claims, characterized in that the base material has a density that deviates from the density of the coloring substances, wherein the density of the base material is preferably 2 to 6 g/cm³, preferably 2 to 3 g/cm³, and/or the density of the one or more coloring substances is from 3 to 13 g/cm³, preferably from 4 to 8 g/cm³.

8. The process according to at least one of the preceding claims, characterized in that the base material and/or the coloring substances have a median particle size D50 of from 0.03 to 100 μm, preferably from 1 to 20 μm, as determined by means of static and/or dynamic light scattering.

9. The process according to at least one of the preceding claims, characterized in that the base material has the following particle size distribution: a D10 of 0.2*D50, preferably 0.5*D50, and a D90 of 4*D50, preferably 2*D50.

10. The process according to at least one of the preceding claims, characterized in that said color pigment particles have a particle size distribution of D10 of 0.1*D50, preferably 0.4*D50 and a D90 of 5*D50, preferably 2*D50.

11. The process according to at least one of the preceding claims, characterized in that the suspension further includes additives, preferably wetting agents and/or dispersants.

12. The process according to at least one of the preceding claims, characterized in that the process according to the invention further includes a step of infiltrating the blank.

13. The process according to at least one of the preceding claims, characterized in that the coloring substances are encapsulated.

14. A blank for producing dental restorations, obtainable by a process according to at least one of claims 1 to 13.

15. Use of a suspension for producing a dental restoration with a color gradient, characterized in that said suspension includes:
i) a liquid dispersant,
ii) a base material, and
iii) one or more coloring substances.

16. The use according to at least one of the preceding claims, characterized in that said dental restoration includes an inlay, onlay, table top, veneer, crowns, partial crowns, implant-supported denture, or a bridge.

17. Use of the blank according to claim 14 for producing dental restorations.
